(19) United States
(12) Patent Application Publication
(10) Pub. No.: US 2022/0160457 A1
(43) Pub. Date: May 26, 2022

(54) BIOPSY DEVICE MARKER AND RELATED METHODS

(71) Applicant: Wake Forest University Health Sciences, Winston-Salem, NC (US)

(72) Inventors: Clifford Howard, Winston-Salem, NC (US); Jordan Eugene Forte, Winston-Salem, NC (US); Margarita Arakelyan-Peters Berwick, Gainsville, FL (US); Nicole Levi, Lexington, NC (US)

(21) Appl. No.: 17/511,864

(22) Filed: Oct. 27, 2021

Related U.S. Application Data
(60) Provisional application No. 63/106,163, filed on Oct. 27, 2020.

(51) Int. Cl. A61B 90/00

(52) U.S. Cl. CPC A61B 90/39; A61B 2090/3908; A61B 2090/3987

(57) ABSTRACT

A biopsy marker device includes: a biopsy marker; and an encapsulation member surrounding the biopsy marker. The encapsulation member comprising a compressible material that is configured to move between a compressed position around the biopsy marker and an expanded position post-deployment.

### FIELD OF THE INVENTION

The present invention relates to biopsy device markers and related methods, more specifically to an encapsulated biopsy marker useful as a breast biopsy device marker and implantation methods for the same.

### BACKGROUND

Biopsy markers are used during the diagnosis of breast cancer. In particular, after a core needle biopsy procedure, metallic markers, typically 1×3 mm, are placed within biopsy cavities to identify lesions for surgical excision and to serve as references in future imaging follow-ups. However, a frequent complication is marker displacement from the original site of placement. Marker displacement, ranging from 1 to 6 cm, occurs in approximately 13-28% of breast biopsies. Often, this observed migration may be a consequence of imprecise marker deployment or the “accordion effect,” where the post-biopsy breast decompression and resulting tissue re-expansion pushes the marker from the biopsy cavity. Consequently, biopsied lesions found to be malignant become subject to an increased risk of incomplete excision during surgery. Although current efforts for minimizing migration have focused on developing marker encasement materials that fill the cavity space to preclude movement, clinically significant levels of marker displacement have persisted. Notably, studies suggest that the imperfect placement of these encased markers within a biopsy cavity may actually result in increased migration along biopsy tracts.

Therefore, there is a need for markers and marker deployment that ensures accurate marker placement to reduce or prevent post-biopsy marker migration.

### SUMMARY OF EMBODIMENTS OF THE INVENTION

According to some embodiments, a biopsy marker device includes: a biopsy marker; and an encapsulation member surrounding the biopsy marker, the encapsulation member comprising a compressible material that is configured to move between a compressed position around the biopsy marker and an expanded position post-deployment.

In some embodiments, the encapsulation member comprises poly(1,8-octanediol-co-citric acid) (POC).

In some embodiments, the biopsy marker comprises a metallic material.

In some embodiments, the encapsulation member is formed from a pre-poly(1,8-octanediol-co-citric acid) (POC) polymer that is post-polymerized around the biopsy marker.

In some embodiments, the pre-poly(1,8-octanediol-co-citric acid) (POC) polymer is synthesized from 1,8-octanediol and citric acid.

In some embodiments, the pre-poly(1,8-octanediol-co-citric acid) (POC) polymer is thermally post-polymerized.

In some embodiments, the encapsulation member expands by at least 200% or at least 300% or at least 350% from the compressed position to the expanded position post-deployment.

According to some embodiments, a deployment device includes a retractable marker sheath having a distal end for receiving a biopsy marker device therein; and a stopper configured to hold the biopsy marker device in the distal end of the retractable marker sheath; wherein the retractable marker sheath is movable between a first position in which the biopsy marker device is in the retractable marker sheath, and a second position in which the retractable marker sheath moves toward the stopper to expose the biopsy marker and deploy the biopsy marker in tissue.

In some embodiments, the stopper comprises a rod the extends through the sheath has a length that is less than a length of the sheath such that, in the first position, the sheath comprises a hollow cylinder end for receiving the biopsy marker device therein.

In some embodiments, the device includes a handle connected to the sheath and configured to move the sheath from the first position to the second position.

In some embodiments, the device includes a biasing member configured to bias the retractable marker sheath in the first position.

In some embodiments, the device includes the deployment device is configured to receive a biopsy marker device as described herein.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a digital image of a biopsy marker device according to some embodiments of the invention.

FIGS. 2 and 3 are schematic diagrams of a deployment device for the biopsy marker of FIG. 1 in a pre-deployment (FIG. 2) and a post-deployment (FIG. 3) configuration according to some embodiments of the invention.

FIG. 4 are images of an unassembled deployment device according to some embodiments of the invention.

FIGS. 5A-5B are a images of an assembled deployment device according to some embodiments of the invention.

FIG. 6 is an end view of an aligned plastic fitting/trigger or handle and outer casing according to some embodiments of the invention.

FIG. 7 is as assembled clip and plastic end plug of the deployment device according to some embodiments of the invention.

FIGS. 8 and 9 are unassembled and assembled views, respectively, of the end clip and trigger/handle according to some embodiments of the invention.

FIG. 10 is a clock-wise rotated clip that is ready for actuating the handle for marker deployment according to some embodiments of the invention.

FIG. 11 is a counter-clockwise-rotated clip indicating that the device has been used and the handle actuated according to some embodiments of the invention.

FIG. 12 is the encapsulation member in a compressed position according to some embodiments of the invention.

FIG. 13 is the encapsulation member in an expanded position according to some embodiments of the invention.

FIGS. 14A-14B is an enlarged view of a sheath (FIG. 14A) in the first position and an end view of the sheath with the marker therein (FIG. 14B) according to some embodiments of the invention.

FIGS. 15A-15B is the sheath of FIGS. 14A-14B in the second, retracted position with the marker released for deployment (FIG. 15A) and an end view of the marker post-deployment (FIG. 15B).

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

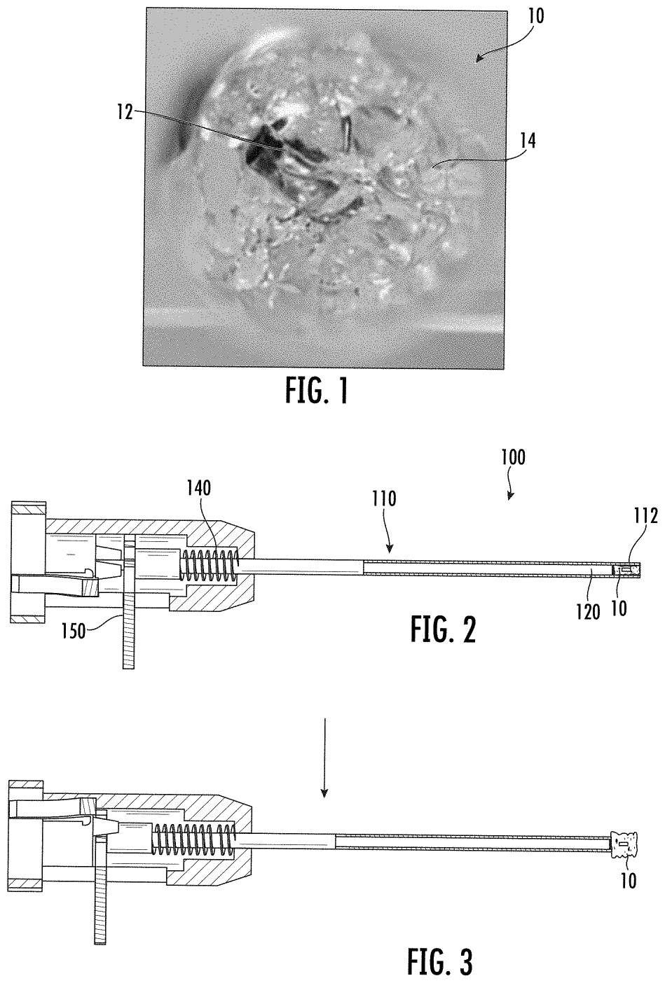

As illustrated in FIG. 1, a biopsy marker device 10 includes a biopsy marker 12 and an encapsulation member 14 surrounding the biopsy marker 12. The encapsulation member 14 comprising a compressible material that is configured to move between a compressed position around the biopsy marker and an expanded position. The expanded position is typically achieved post-deployment in the tissue of a subject. In some embodiments and as illustrated in FIG. 1, the encapsulation member 14 has a surface topology that includes a rough surface, such as local valleys and peaks, which may be configured for more stable placement and less movement when implanted in tissue. As illustrated, the surface topology is irregular. Moreover, when deployed in a tissue cavity, the encapsulation member 14 may expand to fit a tissue cavity.

In some embodiments, the encapsulation member comprises poly(1,8-octanediol-co-citric acid) (POC). The biopsy marker comprises a metallic material. In some embodiments, the encapsulation member is formed from a pre-poly(1,8-octanediol-co-citric acid)(pre-POC) polymer that is post-polymerized around the biopsy marker. The pre-poly(1,8-octanediol-co-citric acid) (POC) polymer may be synthesized from 1,8-octanediol and citric acid. In some embodiments, the pre-poly(1,8-octanediol-co-citric acid) (POC) polymer is thermally post-polymerized.

The encapsulation member may be configured to expand by at least 200% or at least 300% or at least 350% from the compressed position to the expanded position post-deployment.

As shown in FIGS. 2 and 3, a deployment device 100 includes a retractable marker sheath 110 having a distal end 112 for receiving a biopsy marker device 10 therein. A stopper 120, such as a rod that extends through the sheath 110, is configured to hold the biopsy marker device 10 in the distal end 112 of the retractable marker sheath 110, The retractable marker sheath 110 is movable between a first position, as shown in FIG. 2, in which the biopsy marker device 10 is in the retractable marker sheath 110, and a second position, as shown in FIG. 3, in which the retractable marker sheath 110 moves toward the stopper 120 to expose the biopsy marker 10 and deploy the biopsy marker 10 in tissue. The deployment device 100 includes a handle 150 operatively connected to the sheath 110 and configured to move the sheath 110 from the first position in FIG. 2 to the second position in FIG. 3, and a biasing member or spring 140 that is configured to bias the sheath 110 in the pre-deployment, first position in which the biopsy marker 10 is in the sheath 110. The rod 120 has a length that is less than a length of the sheath such that, in the first position of FIG. 2, the sheath 110 comprises a hollow cylinder end for receiving the biopsy marker device 10 therein.

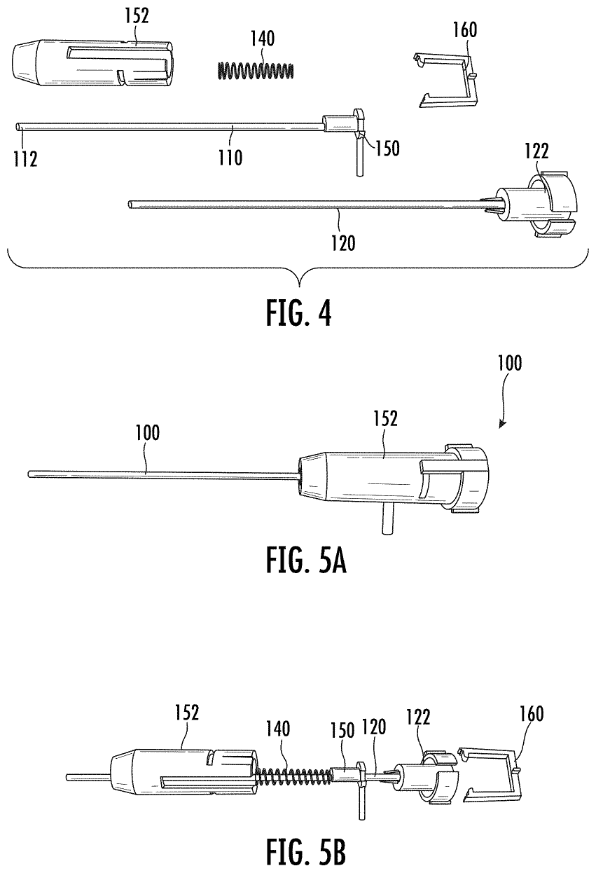

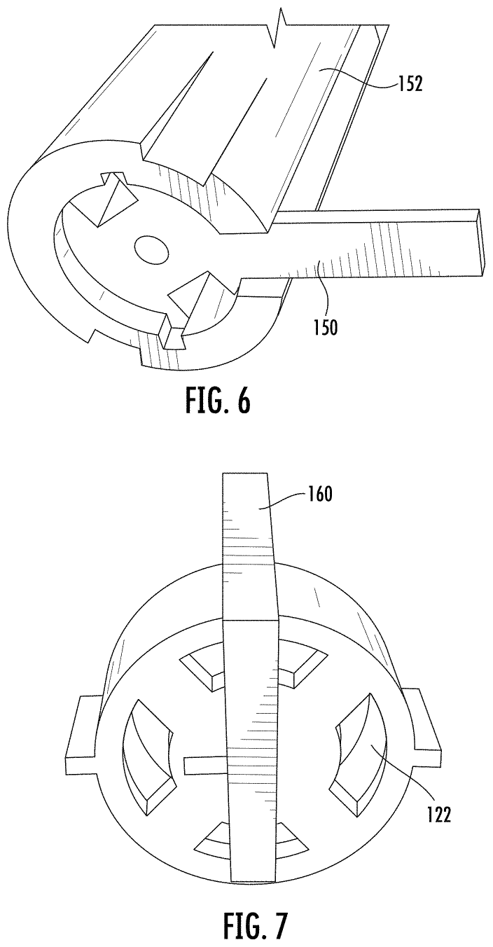

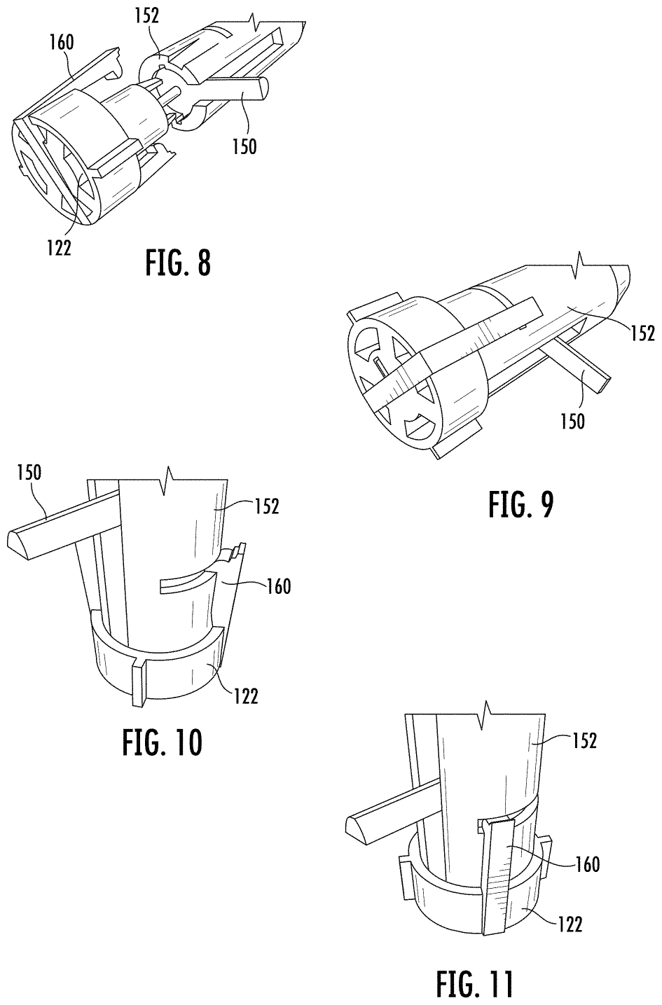

Additional images of the deployment device 100, including partial views of the device 100, are shown in FIGS. 4, 5A-5B, and 6-12. A disassembled deployment device 100 is shown in FIG. 4, including an outer casing 152, a stainless steel hypodermic tube and plastic fitting/trigger (i.e., sheath 110 and handle 150), a compression spring 140, a stainless steel rod and plastic end plug (i.e., stopper 120 and end plug 122, and a clip 160 for holding the components in position as shown in FIG. 5A. An exploded view of a partially assembled device is shown in FIG. 5B. FIG. 6 is an end view of an aligned plastic fitting/trigger or handle 150 and outer casing 512. FIG. 7 is as assembled clip 160 and plastic end plug 122 of the deployment device. FIGS. 8 and 9 are unassembled and assembled views, respectively, of the end clip 169 and trigger/handle 150. The trigger mechanism is configured to rotate (as opposed to button-triggers or triggers relying on pushing/pulling device components). The rotating trigger mechanism utilizes rotation motion that occurs in an axis perpendicular to the axis of deployment. In some embodiment, the rotational trigger mechanism minimizes the likelihood of accidentally moving the device in a way that would affect the location of marker placement because the actuation of the rotational trigger mechanism is perpendicular to the axis of deployment, which may minimize human error.

FIG. 10 is a clock-wise rotated clip 160 that is ready for actuating the handle 150 for marker deployment. FIG. 11 is a counter-clockwise-rotated clip 160 indicating that the device has been used and the handle 150 actuated.

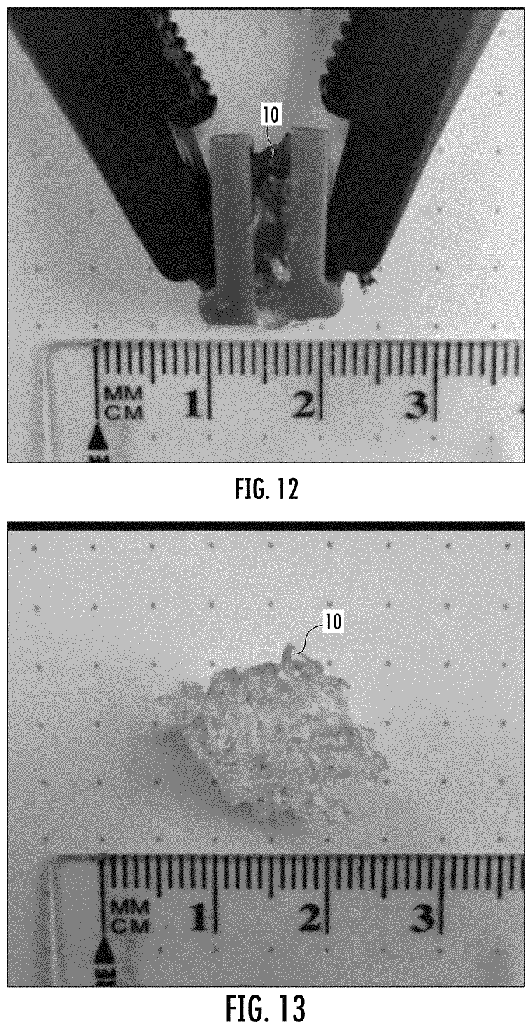

FIG. 12 is the encapsulation member in a compressed position, and FIG. 13 is the encapsulation member in an expanded position. As illustrated, the encapsulation member is a POC sponge or mesh that is compressed to 3 mm in FIG. 12 and is expanded to 20 mm after 30 seconds of decompression in FIG. 13.

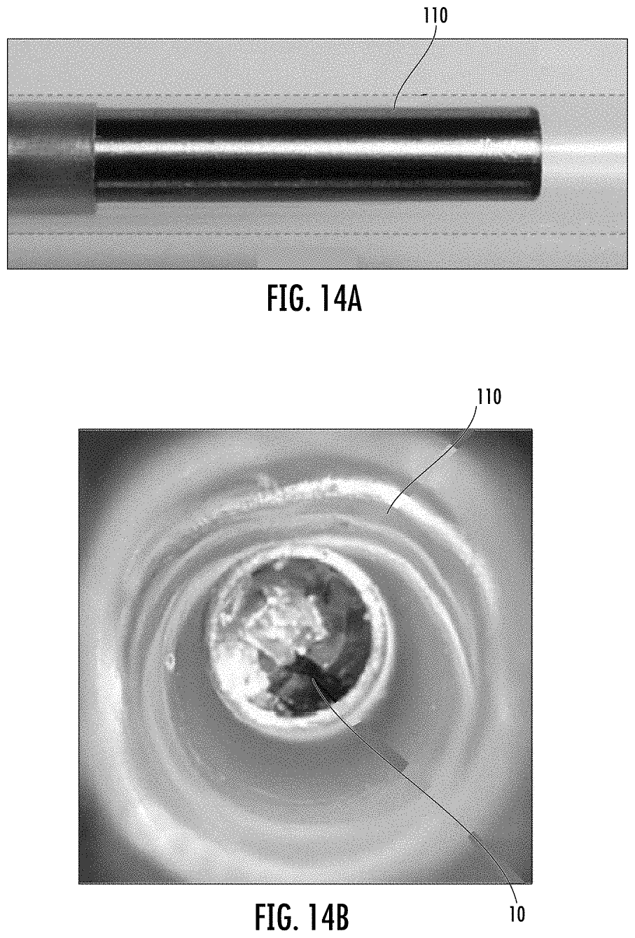

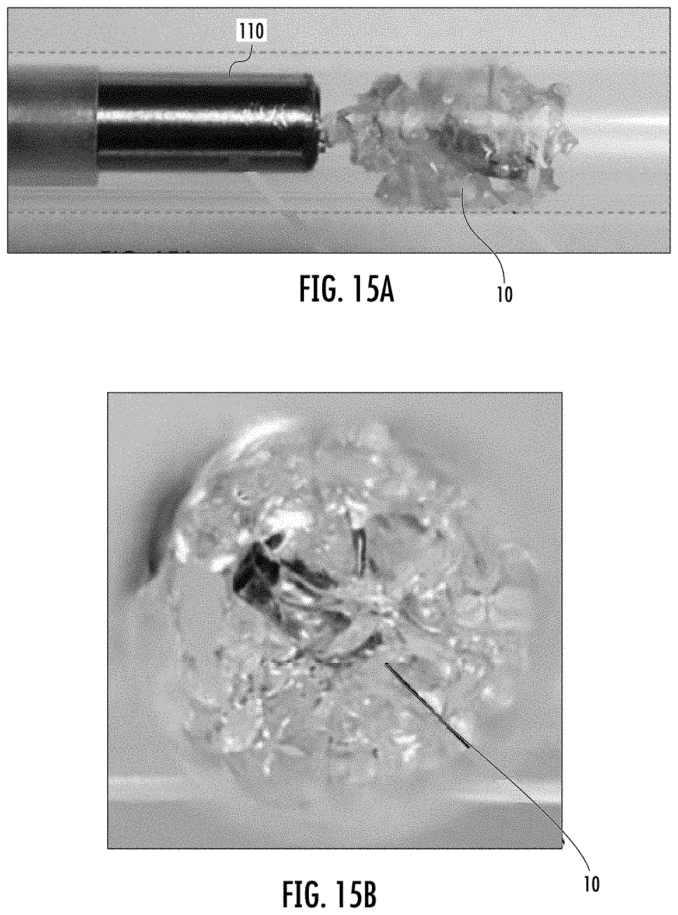

FIG. 14A is an enlarged side view of a sheath 110 in the first position and FIG. 14B is an end view of the sheath 110 with the marker 10 therein. FIGS. 15A-15B is the sheath 110 of FIG. 14 in the second, retracted position with the marker 10 released for deployment in a side view (FIG. 15A) and an end view (FIG. 15B) of the marker post-deployment.

In some embodiments, a pre-poly(1,8-octanediol-co-citric acid) (POC) polymer was synthesized from equimolar amounts of 1,8-octanediol and citric acid and then post-polymerized over high heat in the presence of 1×3 mm marker analogues to yield mesh-encapsulated biopsy markers. Deployment device components were designed using the Tinkercad 3D online platform and printed using polylactic acid (PLA) polymer on an Ultimaker 3 Extended 3D printer. The prototype marker was evaluated for its self-expanding properties by calculating its cross-sectional area pre- and post-deployment from the device. The 3D-printed device was then assessed for placement consistency and accuracy against standard deployment mechanisms by comparing unencapsulated marker ejection distances. The device and marker may be further evaluated in ballistic gel and pork belly tissue models.

The POC-encapsulated markers were found to self-expand to a 373% increase in cross-sectional area following deployment from a compressed state. Additionally, marker ejection distances using the prototype device indicated significantly improved accuracy and consistency when compared to an industry standard device (5.7±1.4 mm and 44.8±16.6 mm, respectively, P<0.001). Qualitative assessment of ballistic gel studies indicated reduced marker rotation following deployment from the device compared to industry standard devices, further supporting the improved placement precision provided by the marker and marker deployment device.

Accordingly, POC-coated markers may have significant potential to resist migration post-biopsy, and the deployment mechanism with retractable sheath may improve marker placement accuracy and consistency compared to current devices.

Although the biopsy marker device is described herein as a POC encapsulated device, it should be understood that any expandable polymeric material may be used, including collagen polymers, poly-ethylene glycol (i.e PEG) hydrogels, and Beta-glucan gels. Pinkney, David M., Mirek Mychajlowycz, and Biren A. Shah. “A prospective comparative study to evaluate the displacement of four commercially available breast biopsy markers.” The British journal of radiology 89.1065 (2016): 20160149. The biopsy marker may be formed of titanium or steel, although any suitable marker material may be used.

The foregoing is illustrative of the present invention and is not to be construed as limiting thereof Although a few example embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A biopsy marker device comprising:
a biopsy marker; and
an encapsulation member surrounding the biopsy marker, the encapsulation member comprising a compressible material that is configured to move between a compressed position around the biopsy marker and an expanded position post-deployment.

2. The biopsy marker device of claim 1, wherein the encapsulation member comprises poly(1,8-octanediol-co-citric acid) (POC).

3. The biopsy marker device of claim 1, wherein the biopsy marker comprises a metallic material.

4. The biopsy marker device of claim 1, wherein the encapsulation member is formed from a pre-poly(1,8-octanediol-co-citric acid) (POC) polymer that is post-polymerized around the biopsy marker.

5. The biopsy marker device of claim 4, wherein the pre-poly(1,8-octanediol-co-citric acid) (POC) polymer is synthesized from 1,8-octanediol and citric acid.

6. The biopsy marker device of claim 4, wherein the pre-poly(1,8-octanediol-co-citric acid) (POC) polymer is thermally post-polymerized.

7. The biopsy marker device of claim 1, wherein the encapsulation member expands by at least 200% or at least 300% or at least 350% from the compressed position to the expanded position post-deployment.

8. The biopsy marker device of claim 1, wherein the encapsulation member comprises a surface topology having peaks and valleys configured for increased stability in use deployed in tissue.

9. A deployment device comprising:
a retractable marker sheath having a distal end for receiving a biopsy marker device therein; and
a stopper configured to hold the biopsy marker device in the distal end of the retractable marker sheath;
wherein the retractable marker sheath is movable between a first position in which the biopsy marker device is in the retractable marker sheath, and a second position in which the retractable marker sheath moves toward the stopper to expose the biopsy marker and deploy the biopsy marker in tissue.

10. The deployment device of claim 9, wherein the stopper comprises a rod the extends through the sheath has a length that is less than a length of the sheath such that, in the first position, the sheath comprises a hollow cylinder end for receiving the biopsy marker device therein.

11. The deployment device of claim 9, further comprising a handle connected to the sheath and configured to move the sheath from the first position to the second position.

12. The deployment device of claim 10, further comprising a biasing member configured to bias the retractable marker sheath in the first position.

13. The deployment device of claim 11, wherein the deployment device is configured to receive a biopsy marker device.
